(19) United States
(12) Patent Application Publication
(10) Pub. No.: US 2021/0382545 A1
(43) Pub. Date: Dec. 9, 2021

(54) HIGH-DEFINITION ELECTRICAL STIMULATION GARMENT FOR EVOKING REALISTIC SOMATOSENSATION

(71) Applicant: Battelle Memorial Institute, Columbus, OH (US)

(72) Inventors: Samuel Colachis, Columbus, OH (US); Andrew Sweeney, Columbus, OH (US); Richard Brooks, Columbus, OH (US)

(21) Appl. No.: 17/339,290

(22) Filed: Jun. 4, 2021

Related U.S. Application Data
(60) Provisional application No. 63/035,706, filed on Jun. 6, 2020.
(60) Provisional application No. 63/072,611, filed on Aug. 31, 2020.

(51) Int. Cl. G06F 3/01; A61N 1/04; A61N 1/36

(52) U.S. Cl. CPC G06F 3/011; A61N 1/0484; A61N 1/36034; A61F 9/08; A61N 1/36021; A61N 1/0476; A61N 1/0452; A61N 1/36003

(57) ABSTRACT

A device comprises: an array of electrodes configured to be disposed on a body part; an electrical stimulation transmitter operatively coupled with the array of electrodes; and a data processing module including an electronic processor and a non-transitory storage medium storing spatiotemporal electrical stimulation patterns for generating corresponding somatosensations and further storing instructions readable and executable by the electronic processor to apply a spatiotemporal electrical stimulation pattern stored in the non-transitory storage medium using the electrical stimulation transmitter and the array of electrodes to generate the somatosensation corresponding to the applied spatiotemporal electrical stimulation pattern. The stored patterns may for example include patterns for generating a sensation of skin exposure to a steam vent, a sensation of a weapon charge fire, a sensation of a rotating athletic ring, a sensation of raindrops, a sensation of a crawling arachnid, or a sensation of a shield strike borne by an arm.

This application claims the benefit of U.S. Provisional Application No. 63/072,611 filed Aug. 31, 2020 and titled “HIGH-DEFINITION ELECTRICAL STIMULATION GARMENT FOR EVOKING REALISTIC SOMATOSENSATION”. This application claims the benefit of U.S. Provisional Application No. 63/035,706 filed Jun. 6, 2020 and titled “HIGH-DEFINITION ELECTRICAL STIMULATION GARMENT FOR EVOKING REALISTIC SOMATOSENSATION”. U.S. Provisional Application No. 63/072,611 filed Aug. 31, 2020 and titled “HIGH-DEFINITION ELECTRICAL STIMULATION GARMENT FOR EVOKING REALISTIC SOMATOSENSATION” is incorporated herein by reference in its entirety. U.S. Provisional Application No. 63/035,706 filed Jun. 6, 2020 and titled “HIGH-DEFINITION ELECTRICAL STIMULATION GARMENT FOR EVOKING REALISTIC SOMATOSENSATION” is incorporated herein by reference in its entirety.

### BACKGROUND

The following relates to the virtual reality (VR) system arts, augmented reality (AR) system arts, electronic gaming arts, simulator arts, patient therapy arts, and to like applications.

As technology advances, humans are constantly striving to become more immersed in the virtual activities they perform. Such activities are encompassed by entertainment, gamming, athletics, educations, and more. Virtual reality is an example of a technology that attempts to immerse humans into a virtual world through visual manipulation. There is still a need across these markets to develop technologies that expand user immersion, making it more realistic and convincing.

Certain improvements are disclosed herein.

### BRIEF SUMMARY

In accordance with some illustrative embodiments disclosed herein, a device comprises: an array of electrodes configured to be disposed on a body part; an electrical stimulation transmitter operatively coupled with the array of electrodes; and a data processing module including an electronic processor and a non-transitory storage medium storing spatiotemporal electrical stimulation patterns for generating corresponding somatosensations and further storing instructions readable and executable by the electronic processor to apply a spatiotemporal electrical stimulation pattern stored in the non-transitory storage medium using the electrical stimulation transmitter and the array of electrodes to generate the somatosensation corresponding to the applied spatiotemporal electrical stimulation pattern. In some specific various embodiments, the stored spatiotemporal electrical stimulation patterns include one or more of: (1) a stored spatiotemporal electrical stimulation pattern for generating a sensation of exposure of skin to a steam vent; (2) a stored spatiotemporal electrical stimulation pattern for generating a sensation of a weapon charge fire; (3) a stored spatiotemporal electrical stimulation pattern for generating a sensation of an athletic ring rotating around an arm; (4) a stored spatiotemporal electrical stimulation pattern for generating a sensation of raindrops falling on skin; (5) a stored spatiotemporal electrical stimulation pattern for generating a sensation of a spider or insect (or, more generally, an arachnid) crawling on skin; and/or (6) a stored spatiotemporal electrical stimulation pattern for generating a sensation of a strike on a shield borne by an arm. These are merely non-limiting illustrative examples.

In accordance with some illustrative embodiments disclosed herein, in a device as set forth in the immediately preceding paragraph, the array of electrodes includes at least 100 electrodes.

In accordance with some illustrative embodiments disclosed herein, a device as set forth in either one of the two immediately preceding paragraphs further includes a garment in which the array of electrodes is embedded. The garment may, for example, comprise a sleeve, and/or a legging.

In accordance with some illustrative embodiments disclosed herein, a therapy method includes disposing a garment on a body part experiencing pain, wherein an array of electrodes is embedded in the garment and, with the garment disposed on the body part, applying an electrical stimulation pattern to the body part using the array of electrodes embedded in the garment.

### BRIEF DESCRIPTION OF THE DRAWINGS

Any quantitative dimensions shown in the drawing are to be understood as non-limiting illustrative examples. Unless otherwise indicated, the drawings are not to scale; if any aspect of the drawings is indicated as being to scale, the illustrated scale is to be understood as non-limiting illustrative example.

FIG. 1 diagrammatically illustrates a virtual reality (VR) or augmented reality (AR) system with capability of delivering somatosensations.

FIG. 2 illustrates a sleeve with an embedded array of electrodes used in some actually performed experiments for delivering somatosensations.

FIG. 3 shows the electrodes channel layout of a sleeve similar to that shown in FIG. 2.

### DETAILED DESCRIPTION

The idea disclosed here is a wearable peripheral for interacting with virtual applications, such as active and passive experiences that will enhance a user's sense of presence with numerous mappable haptic/somatosensory patterns, programable haptic targets, and dynamically correlating multi-modal interactions with stimulated touch feedback. Combined with standard gamming input devices the wearable will enable natural interactions between the user, virtual environment, and virtual objects. The wearable is capable of recording electromyography (EMG) during muscle activity and decoding motor intention to control virtual assets, such as virtual hands. The wearable is also capable of evoking muscle movement through high-definition functional electrical stimulation (FES) that can be paired with virtual events, such as firing a weapon in a virtual shooter game and receiving physical recoil.

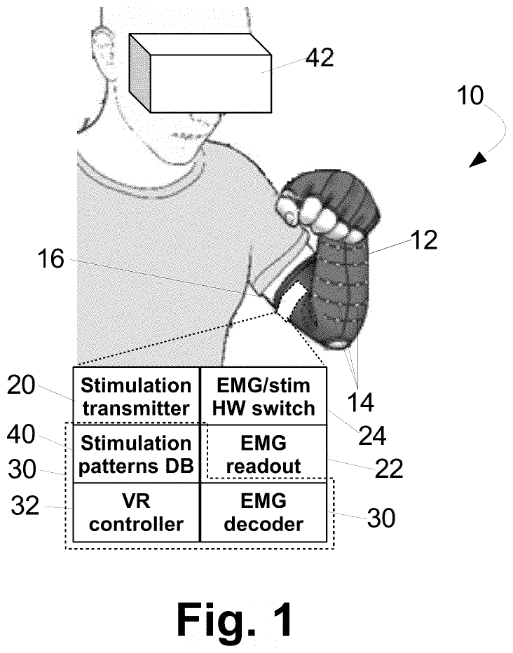

With reference to FIG. 1, a virtual reality (VR) system 10 leverages a wearable garment 12 with electrodes 14 arranged facing the skin so as to contact the skin. (Note that while the electrodes are visible in FIG. 1 for illustration, in some embodiments the electrodes are inside the garment 12 and hence are not visible from the outside). The electrodes include suitable contacts such as hydrogel contacts. For embodiments in which high energy electrical stimulation is to be applied, the hydrogel contacts may optionally comprise sheets extending between the electrodes.

Such wearable garments with electrodes are known for use in electro-neural therapies for medical patients such as stroke victims, patients who are partially or wholly paralyzed due to a spinal cord injury, and so forth. Some suitable embodiments of the garment 12 with electrodes 14 are described, by way of non-limiting illustrative example, in Bouton et al., U.S. Pub. No. 2018/0154133 A1 published Jun. 7, 2018 which is in incorporated herein by reference in its entirety and in Bouton et al., U.S. Pub. No. 2018/0154140 A1 published Jun. 7, 2018 which is in incorporated herein by reference in its entirety and in Bartholomew et al., U.S. Pub. No. 2018/0001086 A1 published Jan. 4, 2018 which is in incorporated herein by reference in its entirety and in Bouton et al., U.S. Pub. No. 2015/0306373 A1 published Oct. 29, 2015 which is in incorporated herein by reference in its entirety.

In the illustrative examples, the wearable garment 12 is a wearable sleeve that is worn on the arm of a person using the VR system 10, as illustrated. However, more generally, the wearable garment may be a legging that is worn on the leg of the person, or a wearable vest or chest band that is worn on the torso and/or abdomen of the person, and/or so forth. Moreover, while use of the wearable garment 12 is beneficial as it provides an efficient way to place a large and dense array of electrodes 14 over an extended area of skin, in some embodiments the array of electrodes 14 may be placed on the skin without the use of a garment serving as a support or substrate for the electrodes 14. For a large array of electrodes, this could be tedious as each electrode would need to be individually placed. If the garment 12 is employed, the placement can be done quickly, simply by putting the garment on. In some embodiments, the garment 12 could take the form of a garment of a type not typically used in clothing persons. For example, the garment 12 carrying the electrodes 14 could be an adhesive tape that is wrapped around the arm of the wearer.

The VR system 10 further includes an electronics module 16, which may be embedded in the wearable garment 12 (as diagrammatically shown) or may be separate from the wearable garment and connected with the electrodes of the garment by suitable wiring. (As an example of the latter, the electronics module 16 could alternatively be embodied as an armband). The electronics module 16 may, for example, comprise electronic mounted on one or more small printed circuit boards, or on a single flexible printed circuit board, or some combination of these arrangements, or so forth.

The illustrative electronics module 16 includes an electrical stimulation transmitter 20 for transmitting electrical stimulation pulses to selected electrodes 14 and an optional electromyography (EMG) readout circuitry 22 for reading EMG signals from the electrodes. The electrical stimulation transmitter 20 typically includes a multichannel simulator allowing for applying electrical stimulation signals with programmed parameters (e.g. amplitude, frequency, waveform, et cetera) to specific electrodes or groups of electrodes. Depending on the magnitude and other characteristics of the electrical stimulation, it may induce functional electrical stimulation (FES) in which muscles are caused to contract by application of the electrical stimulation; or it may induce somatosensations such as a haptic response. The optional EMG readout circuitry 22 typically includes preamplifiers for amplifying the low-strength EMG signals and analog-to-digital (A/D) converters for digitizing the amplified EMG signals. The optional EMG readout circuitry 22 is preferably multichannel so that measured EMG signals are associated to specific electrodes or groups of electrodes. Furthermore, to enable use of the same electrodes 14 for both the optional EMG readout and the electrical stimulation, the illustrative electronics module 16 further includes an EMG/electrical stimulation hardware (HW) switch 24 (e.g., a solid state relay such as a high voltage MOSFET or power transistor) that (1) isolates the EMG readout circuitry 22 from the electrodes 14 and connects the electrical stimulation transmitter 20 during the electrical stimulation phase; and (2) isolates the electrical stimulation transmitter 20 from the electrodes 14 and connects the EMG readout circuitry 22 during the EMG readout phase. Other approaches for implementing both electrical stimulation and EMG readout with the same electrodes are also contemplated, such as use of optoisolators. In another contemplated approach, separate sets of electrodes are used for electrical stimulation and EMG. (In this case, there still may be benefit to time-domain multiplexing between the electrical stimulation and EMG, since the electrical stimulation signals applied during EMG readout will likely interfere with the EMG signal quality). On the other hand, in embodiments that omit EMG readout capability, the EMG readout circuitry 22 and the switch 24 may both suitably be omitted.

As further illustrated in FIG. 1, the illustrative electronics module 16 of the VR system 10 further includes a data processing module 30 which typically includes an electronic processor (e.g. microprocessor or microcontroller) and non-transitory storage medium (details not shown). The non-transitory data storage may, for example, comprise a flash memory, read-only memory (ROM), or other electronic memory (or additionally or alternatively, an optical or magnetic memory such as a miniature hard disk or optical disk). The electronic processor reads and executes instructions stored on the non-transitory storage medium to perform data processing functions as disclosed herein, such as diagrammatically indicated EMG decoder 30 and a virtual reality (VR) controller 32. In a variant embodiment for augmented reality (AR), this may be an AR controller 32. The non-transitory storage medium of the data processing module 30 further stores a somatosensations stimulation database 40 which contains spatiotemporal electrical stimulation patterns for simulating various somatosensations, as will be further described herein.

The optional EMG decoder 30 may be suitably implemented as an artificial neural network (ANN), support vector machine (SVM), or other machine learning (ML) component trained to translate received EMG signals into intended movements of the arm or hand (in the illustrative example of a sleeve garment 12). Training of the EMG decoder 30 is typically done offline, for example by having the wearer perform movements while measuring the EMG signals and then performing supervised training of the ML component using this collected EMG data to optimally train the ML component to output the correct intended movement in response to receiving the corresponding EMG signals. In some typical applications, the EMG readout circuitry 22 and EMG decoder 30 are used to detect muscular actions being done (or attempted) by the person using the VR system 10, and such information may serve as input to the VR controller 32 in order to cause VR elements to respond realistically to those detected muscular actions.

In addition to the foregoing components, the VR system 10 includes a VR headset 42 for present audio-visual elements of the virtual reality environment to the user of the VR system 10. Other sensors (beside the optional EMG readout 22) may be included in the VR headset 42 and/or the wearable garment 12 or elsewhere, such as biometric sensors (e.g., a body temperature sensor, photoplethysmography (PPG) sensor, and/or so forth), accelerometers to track motion of body parts of the user and/or motion of real, physical objects the user interacts with (especially in the case of an AR environment), and so forth. In another variant embodiment, the wearable garment containing the electrodes 14 may comprise two or more garments, e.g. a left-arm sleeve, a right-arm sleeve, a left-leg legging, and a right-leg legging. In such an embodiment, a further short-range radio (e.g. Bluetooth) may be incorporated into each garment in order to allow intercommunication between the various garments (e.g. the left and right sleeves and the left and right leggings) to enable them to operate as a single functional unit. In such an arrangement, only one of these garments may include the electronics module 16, or as previously noted the electronics module 16 may be embodied as a separate component, e.g. a belt-worn module, that is connected with the various garments.

Preferably, the electrodes 14 form a high-density array suitable for optionally measuring high-density electromyography (HDEMG), and suitable for applying complex spatiotemporal electrical stimulation patterns to the wearer's skin in order to simulate complex somatosensations. For example, in some embodiments in which the garment 12 is a sleeve (as illustrated), the sleeve 12 may have 130-160 electrodes, although more or fewer electrodes are also contemplated, e.g. at least 100 electrodes in one specific embodiment. In a functional electrical stimulation (FES) task, the electrical stimulation amplitudes may be on the order of 100-200 volts. By contrast, most somatosensations are generated at lower voltages, thus a higher density of electrodes may be feasible due to the lower electrical stimulation amplitudes typically applied to generate somatosensations.

The VR controller 32 is suitably a conventional VR controller of a type used in conjunction with the VR headset 42 to simulate audio-visual elements of a virtual environment for applications such as VR videogaming, work setting simulators for employee training, enhanced reality audio-video presentations (e.g. movies), and the like. In a variant AR application, the headset 42 is an AR headset which provides partial perception of the real world with superimposed augmented reality features. For example, an AR headset may include eyeglasses, goggles, or the like with transparent lenses that allow the user to see the real world, but in which those transparent lenses have integrated translucent displays that permit superimposing AR elements onto the real world view. In the AR embodiment, the controller 32 is also suitably an AR controller.

In addition, the electrodes 14 of the garment 12 in conjunction with the electrical stimulation transmitter 20 simulate various somatosensations, including but not limited to complex haptic sensations. To this end, the electrodes 14 of the garment 12 generate high-density spatiotemporal electrical stimulation patterns for evoking realistic haptics/somatosensations (and optionally FES where appropriate in the VR environment) and leverages the optional EMG decoding capabilities to decode motor intention to control virtual hands and other virtual assets in the VR environment. The electrical stimulation patterns database 40 stores designed low-current electrical stimulation patterns to evoke specific somatosensory responses. Optionally, the electrical stimulation transmitter 20 may also be used to apply high-current electrical stimulation patterns to evoke muscle contraction through FES. Machine-learning algorithms may optionally be used to decode EMG activity in real-time to control virtual assets.

The VR system 10 may provide further capabilities. For example, biometric sensing may be performed using body temperature sensors, perspiration sensors, or the like incorporated into the sleeve 12 to collect data on the state of the wearer. This data may be used to provide adaptive simulative feedback to the user, and/or integrated in with gaming applications to have the game adapt to the users' biometric state. As a specific example, the closed loop response of the wearer to gaming activity may be thereby monitored. The EMG data, possibly along with such biosensor data, may additionally or alternatively be used to measure (or at least estimate) muscle fatigue, track strength over time (e.g., to detect the wearer becoming tired), or so forth. This data may be used as feedback in gaming or for other applications, such as assessing performance of the wearer in a physiological testing environment such as a stress test or a physical fitness test. Or, in gaming, biosensor data such as heart rate monitoring could be used to monitor exercise conducted using the VR system 10 to provide biofeedback and, for example, to generate audio feedback as to whether the exerciser is drifting out of the target heart rate zone. Likewise, EMG recorded during gaming, sports activity, or entertainers may be used to measure reaction times and other performance metrics during gameplay. Safety-related biometric sensors may also be integrated into the sleeve 12. For example, a glucose monitor may be integrated to detect low or elevated blood sugar levels due to play-induced stress or eating sugary foods between gaming sessions, and a warning provided. This could be especially useful for diabetic gamers.

In a variant embodiment, small air balloons may be disposed on the interior of the sleeve 12, that change the pressure and inflate to get to a certain tightness and then can be used as feedback enhancements simulating contacting of objects in a VR, AR, or other environment. Depending upon the type of object with which contact is being simulated, the electrodes 14 pressed against the skin by the balloon may be energized to produce somatosensations corresponding to a texture of the object.

Conversely, if there is actual contact with a physical object (for example, in an AR system) then flexible force sensors may be included in the sleeve 12 to measure pressure at various places caused by contact with the physical object.

The VR system 10 of FIG. 1 can be used in a wide range of applications, such as fighting simulations in which the user is combating avatars, holograms, simulated robots, simulated military or policing scenarios, or so forth. The electrically generated somatosensations as disclosed herein can enable the user to perceive the feeling of the impact of punches and kicks without the physical contact. Such fighting simulations could also find use in video games, with projected images, or with physical equipment (in an AR application). It will be appreciated that for many of these fight simulation applications, the illustrative sleeve garment 12 may include multiple garments, e.g. left and right sleeves, left and right leggings, a torso vest, a skull cap, et cetera.

The VR system 10 can also be used as a meditation enhancement device. The garment 12 is worn during meditation, and enhances the meditative experience by allowing the user to focus on somatosensations generated by the electrodes 14. The somatosensation may move over time to simulate a feeling of moving energy throughout the body. The somatosensation thus serves as the target of the meditation. Optionally, the electrically generated somatosensations may be generated based on stimulation actual feedback from thought using EEG readings or other measured parameters. As another approach, if the sleeve or other garment 12 includes a heart rate monitor then the somatosensation may be decreased in intensity as the heart rate slows (indicating entry deeper into the meditative state) so as to actively draw the wearer into the meditative state.

Other contemplated applications of the illustrative VR system 10 or a corresponding AR system include use in a haunted house (here the system 10 would be suitable an AR system, either omitting the VR headset 42 or substituting an AR headset headset which provides partial perception of the real world with superimposed augmented reality features. For example, the spatiotemporal electrical stimulation pattern for generating a sensation of a spider (or, more generally, an arachnid) crawling on skin would be effective in a haunted house setting. Similarly, the system 10 implemented as an AR system can be used in laser tag.

In other contemplated applications, audio can be translated into somatosensation. For example, electrical somatosensation haptics can be integrated with music to provide a new form of how to experience the art of music, thereby enhancing the ability of musicians to build the experience and tell the story they want. The approach is also useful for enabling deaf or hard-of-hearing audience members to appreciate the music. In another approach, the electrically generated somatosensations can be used to conduct music (e.g., a baton motion by the human conductor detected by IMU sensors can be translated to somatosensations received by orchestral musicians wearing sleeves 12). Likewise, the somatosensations can operate as a metronome or provide other cues stimulated to the arm. Other types of arts, such as dance or sculpture, could be similarly enhanced. For example, an art museum visitor looking at a tall statue may have a tingling somatosensation when IMUs in the headset 42 indicate the wearer is looking up at the upper portion of the statue, so as to provide an enhanced sensation of the height. In another example, a blind museum visitor may receive electrically induced somatosensations that simulate the shape of the sculpture, enabling the blind visitor to experience “feeling” the sculpture without actually touching it.

The disclosed approach of electrically produced somatosensations using the array of electrodes 14 can also be applied to a handheld gaming controller extension sleeve. Here the electrodes 14 of the sleeve 12 evoke haptic and/or FES stimulation to expand feedback bandwidth. This expands well beyond a conventional vibrating gaming controller. As a use example, the controller may be caused to be dropped with an FES stimulation when the wearer is killed in shooting game. The sleeve 12 could also create somatosensations simulating vibrations, shaking, or other haptics.

In yet another contemplated application, the sleeve 12 (or other garment such as an elastic wristband) with the array of electrodes 14 could serve as a notifications device for various email, text, prioritization received from a cellular telephone or other mobile device with which is it in wired or wireless (e.g. Bluetooth) connection. A “language” of notifications is suitably constructed and stored in the spatiotemporal electrical stimulation patterns database 40, each “word” or “phase” of the language being a somatosensation pattern. Haptics notifications associated with driving are also contemplated, including the language of interpreting different types of notifications: e.g., car approaching, danger, fatigue/sleeping, and/or so forth.

In particular, the somatosensory stimulation aspect has been reduced to practice and tested on multiple able-bodied users. Electrical stimulation patterns have been developed and applied via the electrical stimulation transmitter 20 to evoke the following sensations in a virtual reality dragon shooter game: light, medium, or hard rainfall when passing through a waterfall; weapon charge and reload indication; weapon fire indication (fast and charge shot); enemy fire and hit indication when hit by enemy dragons; and weapon target locking indication. The electrical stimulation transmitter 20 has also been used to evoke the following movements through FES in the virtual reality dragon shooter game: rapid radial deviation during weapon firing to simulate recoil. The stimulation pattern is paired with pulling the trigger on the gaming controller. The VR system has also been used to control a virtual hand with high degrees-of-freedom through decoding EMG using custom machine-learning algorithms. In addition to electrical stimulation patterns for the foregoing somatosensations related to the virtual reality dragon shooter game, electrical stimulation patterns for generating the following additional somatosensations were developed: small to large animal (e.g. spider) crawling on arm; steam vent sensation; athletic rings rotating around the arm; falcon landing on arm; and reaching an arm into fluid.

In another actually constructed game, the sleeve 12 provided light haptic feedback on the inner forearm when the crosshair locks on target and provides FES to create recoil (ulnar deviation) when a shot is fired. Inertial measurement units (IMUs) in the glove portion of the sleeve 12 were used to move the crosshairs based on hand position and bend sensors on the index finger are used to fire the weapon and trigger the recoil FES.

In general, the somatosensations are created using temporally spaced stimulation patterns, also referred to herein as spatiotemporal electrical stimulation patterns. The spatiotemporal electrical stimulation pattern for a given somatosensation is created based on spatial location of active cathode and anode electrodes, stimulation waveform, stimulation amplitude, and stimulation frequency. The design of an electrical stimulation pattern for a given somatosensation is based on a priori knowledge of the spatial location of the sensation, the temporal behavior of the sensation, and the magnitude of the sensation. For example, a spatiotemporal electrical stimulation pattern for athletic rings rotating around the arm are expected to be relatively strong sensations (and hence relatively high amplitude electrical stimulation) that follow a circular path around the arm with a period corresponding to the time interval for one rotation of the ring around the arm. In contrast, the spatiotemporal electrical stimulation pattern for a spider crawling on the arm is expected to be of much lower amplitude (and hence relatively low amplitude electrical stimulation) with the electrical stimulation being applied at discrete points corresponding to footfalls of the eight legs of the spider. The spatiotemporal electrical stimulation pattern for rainfall suitably comprises electrical stimulation applied at discrete locations all over the arm (or over an upper portion of the arm, assuming the rainfall is coming down from above), with the amplitude and rate and area of the electrical stimulation “droplets” being set to simulate the desired “strength” of the rainfall (e.g., light, medium, or hard rainfall).

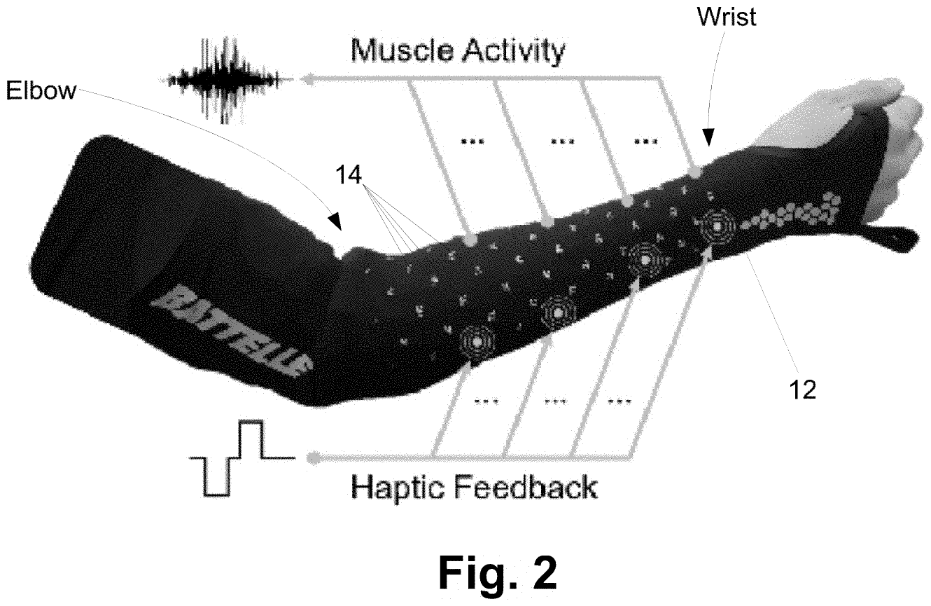

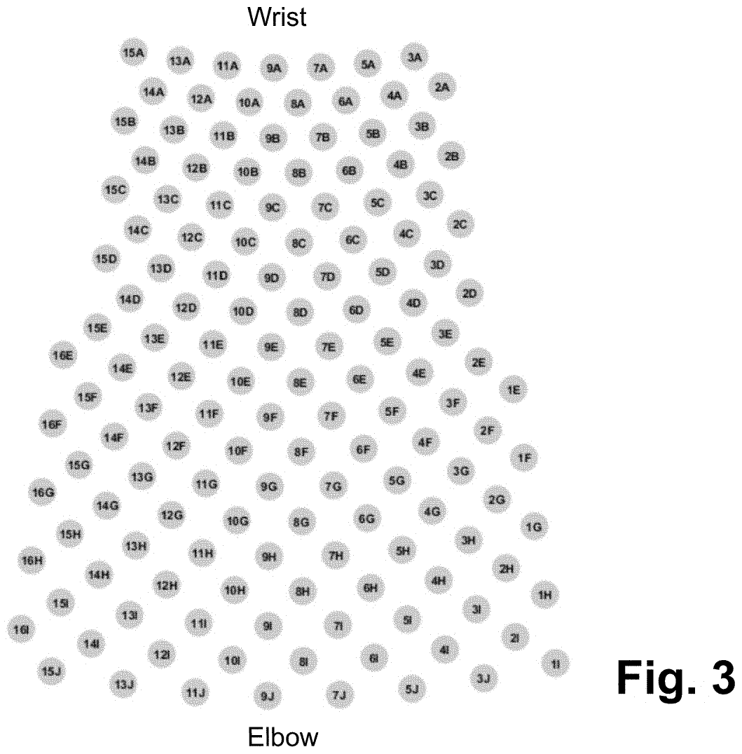

With reference now to FIGS. 2 and 3, some illustrative examples of the actually generated somatosensations are further described. FIG. 2 shows the sleeve 12 used in the actual somatosensation experiments. This sleeve 12 is designed to wrap around the forearm of the user. FIG. 3 shows the electrodes channel layout of the sleeve 12 of FIG. 2, with the sleeve unwrapped to form a planar representation. The electrical stimulation patterns used to generate the somatosensations (corresponding to content of the electrical stimulation patterns database 40 of the diagrammatic representation of FIG. 1) employed a 50 Hz frequency and a stimulation waveform which was a biphasic rectangular function with: Phase I Pulse Width: 500 microseconds; Inter-Pulse Interval: 20 microseconds; Phase II Pulse Width: 1000 microseconds; and Phase III Pulse Width: 5000 microseconds.

In the following, tables are shown of sequences of stimulation patterns in order from the top row (labeled as “wrist” in FIG. 3) to the bottom row (labeled as “elbow” in FIG. 3) for evoking various somatosensations.

SteamVent Sensation

| Positive_Elecs | Negative_Elecs | Amplitude_mA | Duration_s |
|---|---|---|---|
| ‘3A, 5A, 7A’ | ‘2A, 4A, 6A’ | 1 | 0.2 |
| ‘3B, 5B, 7B’ | ‘2B, 4B, 6B’ | 1 | 0.2 |
| ‘3C, 5C, 7C’ | ‘2C, 4C, 6C’ | 1 | 0.2 |
| ‘3D, 5D, 7D’ | ‘2D, 4D, 6D’ | 1 | 0.2 |
| ‘3E, 5E, 7E’ | ‘2E, 4E, 6E’ | 1 | 0.2 |
| ‘3F, 5F, 7F’ | ‘2F, 4F, 6F’ | 1 | 0.2 |
| ‘3G, 5G, 7G’ | ‘2G, 4G, 6G’ | 1 | 0.2 |
| ‘3H, 5H, 7H’ | ‘2H, 4H, 6H’ | 1 | 0.2 |
| ‘3I, 5I, 7I’ | ‘2I, 4I, 6I’ | 1 | 0.2 |
| ‘3A, 5A, 7A’ | ‘2A, 4A, 6A’ | 1 | 0.2 |
| ‘3B, 5B, 7B’ | ‘2B, 4B, 6B’ | 1 | 0.2 |
| ‘3C, 5C, 7C’ | ‘2C, 4C, 6C’ | 1 | 0.2 |
| ‘3D, 5D, 7D’ | ‘2D, 4D, 6D’ | 1 | 0.2 |
| ‘3E, 5E, 7E’ | ‘2E, 4E, 6E’ | 1 | 0.2 |
| ‘3F, 5F, 7F’ | ‘2F, 4F, 6F’ | 1 | 0.2 |
| ‘3G, 5G, 7G’ | ‘2G, 4G, 6G’ | 1 | 0.2 |
| ‘3H, 5H, 7H’ | ‘2H, 4H, 6H’ | 1 | 0.2 |
| ‘3I, 5I, 7I’ | ‘2I, 4I, 6I’ | 1 | 0.2 |
| ‘3A, 5A, 7A’ | ‘2A, 4A, 6A’ | 1 | 0.2 |
| ‘3B, 5B, 7B’ | ‘2B, 4B, 6B’ | 1 | 0.2 |
| ‘3C, 5C, 7C’ | ‘2C, 4C, 6C’ | 1 | 0.2 |
| ‘3D, 5D, 7D’ | ‘2D, 4D, 6D’ | 1 | 0.2 |
| ‘3E, 5E, 7E’ | ‘2E, 4E, 6E’ | 1 | 0.2 |
| ‘3F, 5F, 7F’ | ‘2F, 4F, 6F’ | 1 | 0.2 |
| ‘3G, 5G, 7G’ | ‘2G, 4G, 6G’ | 1 | 0.2 |
| ‘3H, 5H, 7H’ | ‘2H, 4H, 6H’ | 1 | 0.2 |
| ‘3I, 5I, 7I’ | ‘2I, 4I, 6I’ | 1 | 0.2 |

WeaponChargeFire Sensation

| Positive_Elecs | Negative_Elecs | Amplitude_mA | Duration_s |
|---|---|---|---|
| ‘3A, 4A’ | ‘2A, 3B’ | 3 | 0.2 |
| ‘3B, 4B’ | ‘2B, 3C’ | 3 | 0.2 |
| ‘3C, 4C’ | ‘2C, 3D’ | 3 | 0.2 |
| ‘3D, 4D’ | ‘2D, 3E’ | 3 | 0.2 |
| ‘3E, 4E’ | ‘2E, 3F’ | 3 | 0.2 |
| ‘3F, 4F’ | ‘2F, 3G’ | 3 | 0.2 |
| ‘3G, 4G’ | ‘2G, 3H’ | 3 | 0.2 |
| ‘3H, 4H’ | ‘2H, 3I’ | 3 | 0.2 |
| ‘3I, 4I’ | ‘2I, 3J’ | 3 | 0.2 |
| ‘3F, 4E, 4F, 5F’ | ‘7H, 8G, 8H, 9H’ | 5 | 0.1 |

AthleticRingClockwise Sensation

| Positive_Elecs | Negative_Elecs | Amplitude_mA | Duration_s |
|---|---|---|---|
| ‘13G’ | ‘13H’ | 4.5 | 0.05 |
| ‘12G’ | ‘12H’ | 4.5 | 0.05 |
| ‘11G’ | ‘11H’ | 4.5 | 0.05 |
| ‘10G’ | ‘10H’ | 4.5 | 0.05 |
| ‘9G’ | ‘9H’ | 4.5 | 0.05 |
| ‘8G’ | ‘8H’ | 4.5 | 0.05 |
| ‘7G’ | ‘7H’ | 4.5 | 0.05 |
| ‘6G’ | ‘6H’ | 4.5 | 0.05 |
| ‘5G’ | ‘5H’ | 4.5 | 0.05 |
| ‘4G’ | ‘4H’ | 4.5 | 0.05 |
| ‘3G’ | ‘3H’ | 4.5 | 0.05 |
| ‘2G’ | ‘2H’ | 4.5 | 0.05 |
| ‘1G’ | ‘1H’ | 4.5 | 0.05 |
| ‘13G’ | ‘13H’ | 4.5 | 0.05 |
| ‘12G’ | ‘12H’ | 4.5 | 0.05 |
| ‘11G’ | ‘11H’ | 4.5 | 0.05 |
| ‘10G’ | ‘10H’ | 4.5 | 0.05 |
| ‘9G’ | ‘9H’ | 4.5 | 0.05 |
| ‘8G’ | ‘8H’ | 4.5 | 0.05 |
| ‘7G’ | ‘7H’ | 4.5 | 0.05 |
| ‘6G’ | ‘6H’ | 4.5 | 0.05 |
| ‘5G’ | ‘5H’ | 4.5 | 0.05 |
| ‘4G’ | ‘4H’ | 4.5 | 0.05 |
| ‘3G’ | ‘3H’ | 4.5 | 0.05 |
| ‘2G’ | ‘2H’ | 4.5 | 0.05 |
| ‘1G’ | ‘1H’ | 4.5 | 0.05 |
| ‘13G’ | ‘13H’ | 4.5 | 0.05 |
| ‘12G’ | ‘12H’ | 4.5 | 0.05 |
| ‘11G’ | ‘11H’ | 4.5 | 0.05 |
| ‘10G’ | ‘10H’ | 4.5 | 0.05 |
| ‘9G’ | ‘9H’ | 4.5 | 0.05 |
| ‘8G’ | ‘8H’ | 4.5 | 0.05 |
| ‘7G’ | ‘7H’ | 4.5 | 0.05 |
| ‘6G’ | ‘6H’ | 4.5 | 0.05 |
| ‘5G’ | ‘5H’ | 4.5 | 0.05 |
| ‘4G’ | ‘4H’ | 4.5 | 0.05 |
| ‘3G’ | ‘3H’ | 4.5 | 0.05 |
| ‘2G’ | ‘2H’ | 4.5 | 0.05 |
| ‘1G’ | ‘1H’ | 4.5 | 0.05 |
| ‘13G’ | ‘13H’ | 4.5 | 0.05 |
| ‘12G’ | ‘12H’ | 4.5 | 0.05 |
| ‘11G’ | ‘11H’ | 4.5 | 0.05 |
| ‘10G’ | ‘10H’ | 4.5 | 0.05 |
| ‘9G’ | ‘9H’ | 4.5 | 0.05 |
| ‘8G’ | ‘8H’ | 4.5 | 0.05 |
| ‘7G’ | ‘7H’ | 4.5 | 0.05 |
| ‘6G’ | ‘6H’ | 4.5 | 0.05 |
| ‘5G’ | ‘5H’ | 4.5 | 0.05 |
| ‘4G’ | ‘4H’ | 4.5 | 0.05 |
| ‘3G’ | ‘3H’ | 4.5 | 0.05 |
| ‘2G’ | ‘2H’ | 4.5 | 0.05 |
| ‘1G’ | ‘1H’ | 4.5 | 0.05 |
| ‘13G’ | ‘13H’ | 4.5 | 0.05 |
| ‘12G’ | ‘12H’ | 4.5 | 0.05 |
| ‘11G’ | ‘11H’ | 4.5 | 0.05 |
| ‘10G’ | ‘10H’ | 4.5 | 0.05 |
| ‘9G’ | ‘9H’ | 4.5 | 0.05 |
| ‘8G’ | ‘8H’ | 4.5 | 0.05 |
| ‘7G’ | ‘7H’ | 4.5 | 0.05 |
| ‘6G’ | ‘6H’ | 4.5 | 0.05 |
| ‘5G’ | ‘5H’ | 4.5 | 0.05 |
| ‘4G’ | ‘4H’ | 4.5 | 0.05 |
| ‘3G’ | ‘3H’ | 4.5 | 0.05 |
| ‘2G’ | ‘2H’ | 4.5 | 0.05 |
| ‘1G’ | ‘1H’ | 4.5 | 0.05 |
| ‘13G’ | ‘13H’ | 4.5 | 0.05 |
| ‘12G’ | ‘12H’ | 4.5 | 0.05 |
| ‘11G’ | ‘11H’ | 4.5 | 0.05 |
| ‘10G’ | ‘10H’ | 4.5 | 0.05 |
| ‘9G’ | ‘9H’ | 4.5 | 0.05 |
| ‘8G’ | ‘8H’ | 4.5 | 0.05 |
| ‘7G’ | ‘7H’ | 4.5 | 0.05 |
| ‘6G’ | ‘6H’ | 4.5 | 0.05 |
| ‘5G’ | ‘5H’ | 4.5 | 0.05 |
| ‘4G’ | ‘4H’ | 4.5 | 0.05 |
| ‘3G’ | ‘3H’ | 4.5 | 0.05 |
| ‘2G’ | ‘2H’ | 4.5 | 0.05 |
| ‘1G’ | ‘1H’ | 4.5 | 0.05 |
| ‘13G’ | ‘13H’ | 4.5 | 0.05 |
| ‘12G’ | ‘12H’ | 4.5 | 0.05 |
| ‘11G’ | ‘11H’ | 4.5 | 0.05 |
| ‘10G’ | ‘10H’ | 4.5 | 0.05 |
| ‘9G’ | ‘9H’ | 4.5 | 0.05 |
| ‘8G’ | ‘8H’ | 4.5 | 0.05 |
| ‘7G’ | ‘7H’ | 4.5 | 0.05 |
| ‘6G’ | ‘6H’ | 4.5 | 0.05 |
| ‘5G’ | ‘5H’ | 4.5 | 0.05 |
| ‘4G’ | ‘4H’ | 4.5 | 0.05 |
| ‘3G’ | ‘3H’ | 4.5 | 0.05 |
| ‘2G’ | ‘2H’ | 4.5 | 0.05 |
| ‘1G’ | ‘1H’ | 4.5 | 0.05 |
| ‘13G’ | ‘13H’ | 4.5 | 0.05 |
| ‘12G’ | ‘12H’ | 4.5 | 0.05 |
| ‘11G’ | ‘11H’ | 4.5 | 0.05 |
| ‘10G’ | ‘10H’ | 4.5 | 0.05 |
| ‘9G’ | ‘9H’ | 4.5 | 0.05 |
| ‘8G’ | ‘8H’ | 4.5 | 0.05 |
| ‘7G’ | ‘7H’ | 4.5 | 0.05 |
| ‘6G’ | ‘6H’ | 4.5 | 0.05 |
| ‘5G’ | ‘5H’ | 4.5 | 0.05 |
| ‘4G’ | ‘4H’ | 4.5 | 0.05 |
| ‘3G’ | ‘3H’ | 4.5 | 0.05 |
| ‘2G’ | ‘2H’ | 4.5 | 0.05 |
| ‘1G’ | ‘1H’ | 4.5 | 0.05 |
| ‘13G’ | ‘13H’ | 4.5 | 0.05 |
| ‘12G’ | ‘12H’ | 4.5 | 0.05 |
| ‘11G’ | ‘11H’ | 4.5 | 0.05 |
| ‘10G’ | ‘10H’ | 4.5 | 0.05 |
| ‘9G’ | ‘9H’ | 4.5 | 0.05 |
| ‘8G’ | ‘8H’ | 4.5 | 0.05 |
| ‘7G’ | ‘7H’ | 4.5 | 0.05 |
| ‘6G’ | ‘6H’ | 4.5 | 0.05 |
| ‘5G’ | ‘5H’ | 4.5 | 0.05 |
| ‘4G’ | ‘4H’ | 4.5 | 0.05 |
| ‘3G’ | ‘3H’ | 4.5 | 0.05 |
| ‘2G’ | ‘2H’ | 4.5 | 0.05 |
| ‘1G’ | ‘1H’ | 4.5 | 0.05 |
| ‘13G’ | ‘13H’ | 4.5 | 0.05 |
| ‘12G’ | ‘12H’ | 4.5 | 0.05 |
| ‘11G’ | ‘11H’ | 4.5 | 0.05 |
| ‘10G’ | ‘10H’ | 4.5 | 0.05 |
| ‘9G’ | ‘9H’ | 4.5 | 0.05 |
| ‘8G’ | ‘8H’ | 4.5 | 0.05 |
| ‘7G’ | ‘7H’ | 4.5 | 0.05 |
| ‘6G’ | ‘6H’ | 4.5 | 0.05 |
| ‘5G’ | ‘5H’ | 4.5 | 0.05 |
| ‘4G’ | ‘4H’ | 4.5 | 0.05 |
| ‘3G’ | ‘3H’ | 4.5 | 0.05 |
| ‘2G’ | ‘2H’ | 4.5 | 0.05 |
| ‘1G’ | ‘1H’ | 4.5 | 0.05 |

AthleticRingCounterClockwise Sensation

| Positive_Elecs | Negative_Elecs | Amplitude_mA | Duration_s |
|---|---|---|---|
| ‘1G’ | ‘1H’ | 4.5 | 0.05 |
| ‘2G’ | ‘2H’ | 4.5 | 0.05 |
| ‘3G’ | ‘3H’ | 4.5 | 0.05 |
| ‘4G’ | ‘4H’ | 4.5 | 0.05 |
| ‘5G’ | ‘5H’ | 4.5 | 0.05 |
| ‘6G’ | ‘6H’ | 4.5 | 0.05 |
| ‘7G’ | ‘7H’ | 4.5 | 0.05 |
| ‘8G’ | ‘8H’ | 4.5 | 0.05 |
| ‘9G’ | ‘9H’ | 4.5 | 0.05 |
| ‘10G’ | ‘10H’ | 4.5 | 0.05 |
| ‘11G’ | ‘11H’ | 4.5 | 0.05 |
| ‘12G’ | ‘12H’ | 4.5 | 0.05 |
| ‘13G’ | ‘13H’ | 4.5 | 0.05 |
| ‘1G’ | ‘1H’ | 4.5 | 0.05 |
| ‘2G’ | ‘2H’ | 4.5 | 0.05 |
| ‘3G’ | ‘3H’ | 4.5 | 0.05 |
| ‘4G’ | ‘4H’ | 4.5 | 0.05 |
| ‘5G’ | ‘5H’ | 4.5 | 0.05 |
| ‘6G’ | ‘6H’ | 4.5 | 0.05 |
| ‘7G’ | ‘7H’ | 4.5 | 0.05 |
| ‘8G’ | ‘8H’ | 4.5 | 0.05 |
| ‘9G’ | ‘9H’ | 4.5 | 0.05 |
| ‘10G’ | ‘10H’ | 4.5 | 0.05 |
| ‘11G’ | ‘11H’ | 4.5 | 0.05 |
| ‘12G’ | ‘12H’ | 4.5 | 0.05 |
| ‘13G’ | ‘13H’ | 4.5 | 0.05 |
| ‘1G’ | ‘1H’ | 4.5 | 0.05 |
| ‘2G’ | ‘2H’ | 4.5 | 0.05 |
| ‘3G’ | ‘3H’ | 4.5 | 0.05 |
| ‘4G’ | ‘4H’ | 4.5 | 0.05 |
| ‘5G’ | ‘5H’ | 4.5 | 0.05 |
| ‘6G’ | ‘6H’ | 4.5 | 0.05 |
| ‘7G’ | ‘7H’ | 4.5 | 0.05 |
| ‘8G’ | ‘8H’ | 4.5 | 0.05 |
| ‘9G’ | ‘9H’ | 4.5 | 0.05 |
| ‘10G’ | ‘10H’ | 4.5 | 0.05 |
| ‘11G’ | ‘11H’ | 4.5 | 0.05 |
| ‘12G’ | ‘12H’ | 4.5 | 0.05 |
| ‘13G’ | ‘13H’ | 4.5 | 0.05 |
| ‘1G’ | ‘1H’ | 4.5 | 0.05 |
| ‘2G’ | ‘2H’ | 4.5 | 0.05 |
| ‘3G’ | ‘3H’ | 4.5 | 0.05 |
| ‘4G’ | ‘4H’ | 4.5 | 0.05 |
| ‘5G’ | ‘5H’ | 4.5 | 0.05 |
| ‘6G’ | ‘6H’ | 4.5 | 0.05 |
| ‘7G’ | ‘7H’ | 4.5 | 0.05 |
| ‘8G’ | ‘8H’ | 4.5 | 0.05 |
| ‘9G’ | ‘9H’ | 4.5 | 0.05 |
| ‘10G’ | ‘10H’ | 4.5 | 0.05 |
| ‘11G’ | ‘11H’ | 4.5 | 0.05 |
| ‘12G’ | ‘12H’ | 4.5 | 0.05 |
| ‘13G’ | ‘13H’ | 4.5 | 0.05 |
| ‘1G’ | ‘1H’ | 4.5 | 0.05 |
| ‘2G’ | ‘2H’ | 4.5 | 0.05 |
| ‘3G’ | ‘3H’ | 4.5 | 0.05 |
| ‘4G’ | ‘4H’ | 4.5 | 0.05 |
| ‘5G’ | ‘5H’ | 4.5 | 0.05 |
| ‘6G’ | ‘6H’ | 4.5 | 0.05 |
| ‘7G’ | ‘7H’ | 4.5 | 0.05 |
| ‘8G’ | ‘8H’ | 4.5 | 0.05 |
| ‘9G’ | ‘9H’ | 4.5 | 0.05 |
| ‘10G’ | ‘10H’ | 4.5 | 0.05 |
| ‘11G’ | ‘11H’ | 4.5 | 0.05 |
| ‘12G’ | ‘12H’ | 4.5 | 0.05 |
| ‘13G’ | ‘13H’ | 4.5 | 0.05 |
| ‘1G’ | ‘1H’ | 4.5 | 0.05 |
| ‘2G’ | ‘2H’ | 4.5 | 0.05 |
| ‘3G’ | ‘3H’ | 4.5 | 0.05 |
| ‘4G’ | ‘4H’ | 4.5 | 0.05 |
| ‘5G’ | ‘5H’ | 4.5 | 0.05 |
| ‘6G’ | ‘6H’ | 4.5 | 0.05 |
| ‘7G’ | ‘7H’ | 4.5 | 0.05 |
| ‘8G’ | ‘8H’ | 4.5 | 0.05 |
| ‘9G’ | ‘9H’ | 4.5 | 0.05 |
| ‘10G’ | ‘10H’ | 4.5 | 0.05 |
| ‘11G’ | ‘11H’ | 4.5 | 0.05 |
| ‘12G’ | ‘12H’ | 4.5 | 0.05 |
| ‘13G’ | ‘13H’ | 4.5 | 0.05 |
| ‘1G’ | ‘1H’ | 4.5 | 0.05 |
| ‘2G’ | ‘2H’ | 4.5 | 0.05 |
| ‘3G’ | ‘3H’ | 4.5 | 0.05 |
| ‘4G’ | ‘4H’ | 4.5 | 0.05 |
| ‘5G’ | ‘5H’ | 4.5 | 0.05 |
| ‘6G’ | ‘6H’ | 4.5 | 0.05 |
| ‘7G’ | ‘7H’ | 4.5 | 0.05 |
| ‘8G’ | ‘8H’ | 4.5 | 0.05 |
| ‘9G’ | ‘9H’ | 4.5 | 0.05 |
| ‘10G’ | ‘10H’ | 4.5 | 0.05 |
| ‘11G’ | ‘11H’ | 4.5 | 0.05 |
| ‘12G’ | ‘12H’ | 4.5 | 0.05 |
| ‘13G’ | ‘13H’ | 4.5 | 0.05 |
| ‘1G’ | ‘1H’ | 4.5 | 0.05 |
| ‘2G’ | ‘2H’ | 4.5 | 0.05 |
| ‘3G’ | ‘3H’ | 4.5 | 0.05 |
| ‘4G’ | ‘4H’ | 4.5 | 0.05 |
| ‘5G’ | ‘5H’ | 4.5 | 0.05 |
| ‘6G’ | ‘6H’ | 4.5 | 0.05 |
| ‘7G’ | ‘7H’ | 4.5 | 0.05 |
| ‘8G’ | ‘8H’ | 4.5 | 0.05 |
| ‘9G’ | ‘9H’ | 4.5 | 0.05 |
| ‘10G’ | ‘10H’ | 4.5 | 0.05 |
| ‘11G’ | ‘11H’ | 4.5 | 0.05 |
| ‘12G’ | ‘12H’ | 4.5 | 0.05 |
| ‘13G’ | ‘13H’ | 4.5 | 0.05 |
| ‘1G’ | ‘1H’ | 4.5 | 0.05 |
| ‘2G’ | ‘2H’ | 4.5 | 0.05 |
| ‘3G’ | ‘3H’ | 4.5 | 0.05 |
| ‘4G’ | ‘4H’ | 4.5 | 0.05 |
| ‘5G’ | ‘5H’ | 4.5 | 0.05 |
| ‘6G’ | ‘6H’ | 4.5 | 0.05 |
| ‘7G’ | ‘7H’ | 4.5 | 0.05 |
| ‘8G’ | ‘8H’ | 4.5 | 0.05 |
| ‘9G’ | ‘9H’ | 4.5 | 0.05 |
| ‘10G’ | ‘10H’ | 4.5 | 0.05 |
| ‘11G’ | ‘11H’ | 4.5 | 0.05 |
| ‘12G’ | ‘12H’ | 4.5 | 0.05 |
| ‘13G’ | ‘13H’ | 4.5 | 0.05 |
| ‘1G’ | ‘1H’ | 4.5 | 0.05 |
| ‘2G’ | ‘2H’ | 4.5 | 0.05 |
| ‘3G’ | ‘3H’ | 4.5 | 0.05 |
| ‘4G’ | ‘4H’ | 4.5 | 0.05 |
| ‘5G’ | ‘5H’ | 4.5 | 0.05 |
| ‘6G’ | ‘6H’ | 4.5 | 0.05 |
| ‘7G’ | ‘7H’ | 4.5 | 0.05 |
| ‘8G’ | ‘8H’ | 4.5 | 0.05 |
| ‘9G’ | ‘9H’ | 4.5 | 0.05 |
| ‘10G’ | ‘10H’ | 4.5 | 0.05 |
| ‘11G’ | ‘11H’ | 4.5 | 0.05 |
| ‘12G’ | ‘12H’ | 4.5 | 0.05 |
| ‘13G’ | ‘13H’ | 4.5 | 0.05 |

RainDrops Sensation

| Positive_Elecs | Negative_Elecs | Amplitude_mA | Duration |
|---|---|---|---|
| ‘6C’ | ‘7C’ | 5 | 0.1 |
| ‘4D’ | ‘5D’ | 5 | 0.1 |
| ‘2G’ | ‘3G’ | 5 | 0.1 |
| ‘2H’ | ‘3H’ | 5 | 0.1 |
| ‘6H’ | ‘7H’ | 5 | 0.1 |
| ‘2A’ | ‘3A’ | 5 | 0.1 |
| ‘2F’ | ‘3F’ | 5 | 0.1 |
| ‘4F’ | ‘5F’ | 5 | 0.1 |
| ‘6D’ | ‘7D’ | 5 | 0.1 |
| ‘6F’ | ‘7F’ | 5 | 0.1 |
| ‘4H’ | ‘5H’ | 5 | 0.1 |
| ‘4C’ | ‘5C’ | 5 | 0.1 |
| ‘4A’ | ‘5A’ | 5 | 0.1 |
| ‘6G’ | ‘7G’ | 5 | 0.1 |
| ‘6H’ | ‘7H’ | 5 | 0.1 |
| ‘6C’ | ‘7C’ | 5 | 0.1 |
| ‘2F’ | ‘3F’ | 5 | 0.1 |
| ‘2F’ | ‘3F’ | 5 | 0.1 |
| ‘4H’ | ‘5H’ | 5 | 0.1 |
| ‘4E’ | ‘5E’ | 5 | 0.1 |
| ‘6G’ | ‘7G’ | 5 | 0.1 |
| ‘2D’ | ‘3D’ | 5 | 0.1 |
| ‘4E’ | ‘5E’ | 5 | 0.1 |
| ‘4E’ | ‘5E’ | 5 | 0.1 |
| ‘4H’ | ‘5H’ | 5 | 0.1 |
| ‘2H’ | ‘3H’ | 5 | 0.1 |
| ‘4G’ | ‘5G’ | 5 | 0.1 |
| ‘4F’ | ‘5F’ | 5 | 0.1 |
| ‘4B’ | ‘5B’ | 5 | 0.1 |
| ‘2E’ | ‘3E’ | 5 | 0.1 |
| ‘6E’ | ‘7E’ | 5 | 0.1 |
| ‘4F’ | ‘5F’ | 5 | 0.1 |
| ‘4A’ | ‘5A’ | 5 | 0.1 |
| ‘2G’ | ‘3G’ | 5 | 0.1 |
| ‘2D’ | ‘3D’ | 5 | 0.1 |
| ‘4C’ | ‘5C’ | 5 | 0.1 |
| ‘4B’ | ‘5B’ | 5 | 0.1 |
| ‘2H’ | ‘3H’ | 5 | 0.1 |
| ‘2C’ | ‘3C’ | 5 | 0.1 |
| ‘4D’ | ‘5D’ | 5 | 0.1 |
| ‘4G’ | ‘5G’ | 5 | 0.1 |
| ‘4A’ | ‘5A’ | 5 | 0.1 |
| ‘6F’ | ‘7F’ | 5 | 0.1 |
| ‘6A’ | ‘7A’ | 5 | 0.1 |
| ‘6H’ | ‘7H’ | 5 | 0.1 |
| ‘2G’ | ‘3G’ | 5 | 0.1 |
| ‘2B’ | ‘3B’ | 5 | 0.1 |
| ‘2B’ | ‘3B’ | 5 | 0.1 |
| ‘6G’ | ‘7G’ | 5 | 0.1 |
| ‘4D’ | ‘5D’ | 5 | 0.1 |
| ‘2A’ | ‘3A’ | 5 | 0.1 |
| ‘2C’ | ‘3C’ | 5 | 0.1 |
| ‘2E’ | ‘3E’ | 5 | 0.1 |
| ‘6E’ | ‘7E’ | 5 | 0.1 |
| ‘6B’ | ‘7B’ | 5 | 0.1 |
| ‘6D’ | ‘7D’ | 5 | 0.1 |
| ‘6E’ | ‘7E’ | 5 | 0.1 |
| ‘4G’ | ‘5G’ | 5 | 0.1 |
| ‘2A’ | ‘3A’ | 5 | 0.1 |
| ‘6A’ | ‘7A’ | 5 | 0.1 |
| ‘2E’ | ‘3E’ | 5 | 0.1 |
| ‘2D’ | ‘3D’ | 5 | 0.1 |
| ‘2C’ | ‘3C’ | 5 | 0.1 |
| ‘6B’ | ‘7B’ | 5 | 0.1 |
| ‘4B’ | ‘5B’ | 5 | 0.1 |
| ‘6F’ | ‘7F’ | 5 | 0.1 |
| ‘6C’ | ‘7C’ | 5 | 0.1 |
| ‘6A’ | ‘7A’ | 5 | 0.1 |
| ‘6B’ | ‘7B’ | 5 | 0.1 |
| ‘4C’ | ‘5C’ | 5 | 0.1 |
| ‘2B’ | ‘3B’ | 5 | 0.1 |
| ‘6D’ | ‘7D’ | 5 | 0.1 |

Spider Sensation

| Positive_Elecs | Negative_Elecs | Amplitude_mA | Duration_s |
|---|---|---|---|
| ‘3A’ | ‘4A’ | 8 | 0.2 |
| ‘4A’ | ‘3B’ | 8 | 0.2 |
| ‘3B’ | ‘4B’ | 8 | 0.2 |
| ‘4B’ | ‘3C’ | 8 | 0.2 |
| ‘3C’ | ‘4C’ | 8 | 0.2 |
| ‘4C’ | ‘3D’ | 8 | 0.2 |
| ‘3D’ | ‘4D’ | 8 | 0.2 |
| ‘4D’ | ‘3E’ | 8 | 0.2 |
| ‘3E’ | ‘4E’ | 8 | 0.2 |
| ‘4E’ | ‘3F’ | 8 | 0.2 |
| ‘3F’ | ‘4F’ | 8 | 0.2 |
| ‘4F’ | ‘3G’ | 8 | 0.2 |
| ‘3G’ | ‘4G’ | 8 | 0.2 |
| ‘4G’ | ‘3H’ | 8 | 0.2 |
| ‘3H’ | ‘4H’ | 8 | 0.2 |
| ‘4H’ | ‘3I’ | 8 | 0.2 |
| ‘3I’ | ‘4I’ | 8 | 0.2 |
| ‘4I’ | ‘3J’ | 8 | 0.2 |
| ‘3I’ | ‘4I’ | 8 | 0.2 |
| ‘4H’ | ‘3I’ | 8 | 0.2 |
| ‘3H’ | ‘4H’ | 8 | 0.2 |
| ‘4G’ | ‘3H’ | 8 | 0.2 |
| ‘3G’ | ‘4G’ | 8 | 0.2 |
| ‘4F’ | ‘3G’ | 8 | 0.2 |
| ‘3F’ | ‘4F’ | 8 | 0.2 |
| ‘4E’ | ‘3F’ | 8 | 0.2 |
| ‘3E’ | ‘4E’ | 8 | 0.2 |
| ‘4D’ | ‘3E’ | 8 | 0.2 |
| ‘3D’ | ‘4D’ | 8 | 0.2 |
| ‘4C’ | ‘3D’ | 8 | 0.2 |
| ‘3C’ | ‘4C’ | 8 | 0.2 |
| ‘4B’ | ‘3C’ | 8 | 0.2 |
| ‘3B’ | ‘4B’ | 8 | 0.2 |
| ‘4A’ | ‘3B’ | 8 | 0.2 |
| ‘3A’ | ‘4A’ | 8 | 0.2 |

RainDropsMedim Sensation

| Positive_Elecs | Negative_Elecs | Amplitude_mA | Duration_s |
|---|---|---|---|
| ‘6C’ | ‘7C’ | 6 | 0.1 |
| ‘4D’ | ‘5D’ | 6 | 0.1 |
| ‘2G’ | ‘3G’ | 6 | 0.1 |
| ‘2H’ | ‘3H’ | 6 | 0.1 |
| ‘6H’ | ‘7H’ | 6 | 0.1 |
| ‘2A’ | ‘3A’ | 6 | 0.1 |
| ‘2F’ | ‘3F’ | 6 | 0.1 |
| ‘4F’ | ‘5F’ | 6 | 0.1 |
| ‘6D’ | ‘7D’ | 6 | 0.1 |
| ‘6F’ | ‘7F’ | 6 | 0.1 |
| ‘4H’ | ‘5H’ | 6 | 0.1 |
| ‘4C’ | ‘5C’ | 6 | 0.1 |
| ‘4A’ | ‘5A’ | 6 | 0.1 |
| ‘6G’ | ‘7G’ | 6 | 0.1 |
| ‘6H’ | ‘7H’ | 6 | 0.1 |
| ‘6C’ | ‘7C’ | 6 | 0.1 |
| ‘2F’ | ‘3F’ | 6 | 0.1 |
| ‘2F’ | ‘3F’ | 6 | 0.1 |
| ‘4H’ | ‘5H’ | 6 | 0.1 |
| ‘4E’ | ‘5E’ | 6 | 0.1 |
| ‘6G’ | ‘7G’ | 6 | 0.1 |
| ‘2D’ | ‘3D’ | 6 | 0.1 |
| ‘4E’ | ‘5E’ | 6 | 0.1 |
| ‘4E’ | ‘5E’ | 6 | 0.1 |
| ‘4H’ | ‘5H’ | 6 | 0.1 |
| ‘2H’ | ‘3H’ | 6 | 0.1 |
| ‘4G’ | ‘5G’ | 6 | 0.1 |
| ‘4F’ | ‘5F’ | 6 | 0.1 |
| ‘4B’ | ‘5B’ | 6 | 0.1 |
| ‘2E’ | ‘3E’ | 6 | 0.1 |
| ‘6E’ | ‘7E’ | 6 | 0.1 |
| ‘4F’ | ‘5F’ | 6 | 0.1 |
| ‘4A’ | ‘5A’ | 6 | 0.1 |
| ‘2G’ | ‘3G’ | 6 | 0.1 |
| ‘2D’ | ‘3D’ | 6 | 0.1 |
| ‘4C’ | ‘5C’ | 6 | 0.1 |
| ‘4B’ | ‘5B’ | 6 | 0.1 |
| ‘2H’ | ‘3H’ | 6 | 0.1 |
| ‘2C’ | ‘3C’ | 6 | 0.1 |
| ‘4D’ | ‘5D’ | 6 | 0.1 |
| ‘4G’ | ‘5G’ | 6 | 0.1 |
| ‘4A’ | ‘5A’ | 6 | 0.1 |
| ‘6F’ | ‘7F’ | 6 | 0.1 |
| ‘6A’ | ‘7A’ | 6 | 0.1 |
| ‘6H’ | ‘7H’ | 6 | 0.1 |
| ‘2G’ | ‘3G’ | 6 | 0.1 |
| ‘2B’ | ‘3B’ | 6 | 0.1 |
| ‘2B’ | ‘3B’ | 6 | 0.1 |
| ‘6G’ | ‘7G’ | 6 | 0.1 |
| ‘4D’ | ‘5D’ | 6 | 0.1 |
| ‘2A’ | ‘3A’ | 6 | 0.1 |
| ‘2C’ | ‘3C’ | 6 | 0.1 |
| ‘2E’ | ‘3E’ | 6 | 0.1 |
| ‘6E’ | ‘7E’ | 6 | 0.1 |
| ‘6B’ | ‘7B’ | 6 | 0.1 |
| ‘6D’ | ‘7D’ | 6 | 0.1 |
| ‘6E’ | ‘7E’ | 6 | 0.1 |
| ‘4G’ | ‘5G’ | 6 | 0.1 |
| ‘2A’ | ‘3A’ | 6 | 0.1 |
| ‘6A’ | ‘7A’ | 6 | 0.1 |
| ‘2E’ | ‘3E’ | 6 | 0.1 |
| ‘2D’ | ‘3D’ | 6 | 0.1 |
| ‘2C’ | ‘3C’ | 6 | 0.1 |
| ‘6B’ | ‘7B’ | 6 | 0.1 |
| ‘4B’ | ‘5B’ | 6 | 0.1 |
| ‘6F’ | ‘7F’ | 6 | 0.1 |
| ‘6C’ | ‘7C’ | 6 | 0.1 |
| ‘6A’ | ‘7A’ | 6 | 0.1 |
| ‘6B’ | ‘7B’ | 6 | 0.1 |
| ‘4C’ | ‘5C’ | 6 | 0.1 |
| ‘2B’ | ‘3B’ | 6 | 0.1 |
| ‘6D’ | ‘7D’ | 6 | 0.1 |

RainDropsHard Sensation

| Positive_Elecs | Negative_Elecs | Amplitude_mA | Duration_s |
|---|---|---|---|
| ‘6C’ | ‘7C’ | 7 | 0.1 |
| ‘4D’ | ‘5D’ | 7 | 0.1 |
| ‘2G’ | ‘3G’ | 7 | 0.1 |
| ‘2H’ | ‘3H’ | 7 | 0.1 |
| ‘6H’ | ‘7H’ | 7 | 0.1 |
| ‘2A’ | ‘3A’ | 7 | 0.1 |
| ‘2F’ | ‘3F’ | 7 | 0.1 |
| ‘4F’ | ‘5F’ | 7 | 0.1 |
| ‘6D’ | ‘7D’ | 7 | 0.1 |
| ‘6F’ | ‘7F’ | 7 | 0.1 |
| ‘4H’ | ‘5H’ | 7 | 0.1 |
| ‘4C’ | ‘5C’ | 7 | 0.1 |
| ‘4A’ | ‘5A’ | 7 | 0.1 |
| ‘6G’ | ‘7G’ | 7 | 0.1 |
| ‘6H’ | ‘7H’ | 7 | 0.1 |
| ‘6C’ | ‘7C’ | 7 | 0.1 |
| ‘2F’ | ‘3F’ | 7 | 0.1 |
| ‘2F’ | ‘3F’ | 7 | 0.1 |
| ‘4H’ | ‘5H’ | 7 | 0.1 |
| ‘4E’ | ‘5E’ | 7 | 0.1 |
| ‘6G’ | ‘7G’ | 7 | 0.1 |
| ‘2D’ | ‘3D’ | 7 | 0.1 |
| ‘4E’ | ‘5E’ | 7 | 0.1 |
| ‘4E’ | ‘5E’ | 7 | 0.1 |
| ‘4H’ | ‘5H’ | 7 | 0.1 |
| ‘2H’ | ‘3H’ | 7 | 0.1 |
| ‘4G’ | ‘5G’ | 7 | 0.1 |
| ‘4F’ | ‘5F’ | 7 | 0.1 |
| ‘4B’ | ‘5B’ | 7 | 0.1 |
| ‘2E’ | ‘3E’ | 7 | 0.1 |
| ‘6E’ | ‘7E’ | 7 | 0.1 |
| ‘4F’ | ‘5F’ | 7 | 0.1 |
| ‘4A’ | ‘5A’ | 7 | 0.1 |
| ‘2G’ | ‘3G’ | 7 | 0.1 |
| ‘2D’ | ‘3D’ | 7 | 0.1 |
| ‘4C’ | ‘5C’ | 7 | 0.1 |
| ‘4B’ | ‘5B’ | 7 | 0.1 |
| ‘2H’ | ‘3H’ | 7 | 0.1 |
| ‘2C’ | ‘3C’ | 7 | 0.1 |
| ‘4D’ | ‘5D’ | 7 | 0.1 |
| ‘4G’ | ‘5G’ | 7 | 0.1 |
| ‘4A’ | ‘5A’ | 7 | 0.1 |
| ‘6F’ | ‘7F’ | 7 | 0.1 |
| ‘6A’ | ‘7A’ | 7 | 0.1 |
| ‘6H’ | ‘7H’ | 7 | 0.1 |
| ‘2G’ | ‘3G’ | 7 | 0.1 |
| ‘2B’ | ‘3B’ | 7 | 0.1 |
| ‘2B’ | ‘3B’ | 7 | 0.1 |
| ‘6G’ | ‘7G’ | 7 | 0.1 |
| ‘4D’ | ‘5D’ | 7 | 0.1 |
| ‘2A’ | ‘3A’ | 7 | 0.1 |
| ‘2C’ | ‘3C’ | 7 | 0.1 |
| ‘2E’ | ‘3E’ | 7 | 0.1 |
| ‘6E’ | ‘7E’ | 7 | 0.1 |
| ‘6B’ | ‘7B’ | 7 | 0.1 |
| ‘6D’ | ‘7D’ | 7 | 0.1 |
| ‘6E’ | ‘7E’ | 7 | 0.1 |
| ‘4G’ | ‘5G’ | 7 | 0.1 |
| ‘2A’ | ‘3A’ | 7 | 0.1 |
| ‘6A’ | ‘7A’ | 7 | 0.1 |
| ‘2E’ | ‘3E’ | 7 | 0.1 |
| ‘2D’ | ‘3D’ | 7 | 0.1 |
| ‘2C’ | ‘3C’ | 7 | 0.1 |
| ‘6B’ | ‘7B’ | 7 | 0.1 |
| ‘4B’ | ‘5B’ | 7 | 0.1 |
| ‘6F’ | ‘7F’ | 7 | 0.1 |
| ‘6C’ | ‘7C’ | 7 | 0.1 |
| ‘6A’ | ‘7A’ | 7 | 0.1 |
| ‘6B’ | ‘7B’ | 7 | 0.1 |
| ‘4C’ | ‘5C’ | 7 | 0.1 |
| ‘2B’ | ‘3B’ | 7 | 0.1 |
| ‘6D’ | ‘7D’ | 7 | 0.1 |

| | | | |
|---|---|---|---|
| Positive_Elecs | Negative_Elecs | Amplitude_mA | Duration_s |

WeaponFire Sensation

| | | | |
|---|---|---|---|
| ‘4F, 5E, 5F, 6F’ | ‘8H, 9G, 9H, 10H’ | 6 | 0.1 |

ShieldHitLeft Sensation

| | | | |
|---|---|---|---|
| ‘9F, 9G, 10E, 10F’ | ‘9H, 9I, 10G, 10H’ | 4 | 0.2 |

ShieldHitTop Sensation

| | | | |
|---|---|---|---|
| ‘6E, 6F, 7E, 7F’ | ‘6G, 6H, 7G, 7H’ | 4 | 0.2 |

ShieldHitEight Sensation

| | | | |
|---|---|---|---|
| ‘3F, 3G, 4E, 4F ' | ‘3H, 3I, 4G, 4H’ | 6 | 0.2 |

The foregoing are merely illustrative examples, and more generally a wide range of somatosensations may be similarly simulated by constructing suitable spatiotemporal electrical stimulation patterns based on a priori knowledge of the spatial and temporal distribution of the haptic or other sensations on the skin. In general, neuromuscular electrical stimulation (NMES) is applied to evoke movement (corresponding to FES), while low-current stimulation is applied to target receptors in the skin (somatosensory), sometimes referred to as transcutaneous electrical nerve stimulation (TENS).

In some contemplated variant embodiments, which have not yet been reduced to practice, it is contemplated for the somatosensations to be other than haptic sensations. For example, an electrical stimulation of sufficiently high amplitude may generate a pain sensation. While pain sensations might not be acceptable in a gaming setting, for simulations such as VR or AR simulation of a combat situation for training soldiers, imparting pain in response to being struck by a bullet in the VR or AR environment may be acceptable as a means for motivating the soldier-in-training. As another example, a rectangular stimulation pulse that turns (e.g., a rapidly turning stimulation pattern) is expected to produce a “sharp” feeling that could be effective in stimulating somatosensations such as a stab event caused by a sword or knife point, spear, or other pointed weapon. Likewise, a rectangular stimulation pulse that moves rapidly along the arm could simulate a cut event caused by the edge of a sword or knife or other bladed weapon.

As other examples, somatosensations may be applied for medical patients for therapeutic purposes. In these applications, the applied somatosensations are received by the somatosensory system of the patient, but are not necessarily designed to mimic a specific sensory source. Rather, the applied somatosensations may counter numbness, pain, or other discomfort of the patient.

For example, a ganglion cyst is a fluid-filled bump associated with a joint or tendon sheath. Ganglion cysts occurring in facial muscles can cause pain and facial migraines, while ganglion cysts on the arm, hand, or elsewhere can lead to pain, numbness, or other symptoms. In the case of a ganglion cyst on the arm or wrist, the sleeve of FIG. 2 can be used to stimulate the ganglion cyst to provide an anesthetic effect that mitigates pain and numbness. Similarly, a cyst on the ankle or leg could be treated using a legging garment with the array of electrodes 14. In the case of a facial ganglion cyst, the array of electrodes 14 could be disposed on an adhesive patch, or on a face mask (e.g., similar to a ski mask), or the like to contact with the afflicted facial musculature, and energized to provide an anesthetic effect to mitigate the pain of a migraine and potentially prevent them in the future. It is further contemplated that such patient therapy could be deployed in combination with the VR system 10 of FIG. 1 to deliver non-invasive somatosensation treatment in combination with a calming video game or other VR environment (e.g., presenting a peaceful mountain lake environment).

As another example, the illustrative sleeve 12 could be used to treat tennis elbow and/or tendon related pain, muscle cramping, or the like in the arm. The sleeve 12 in this embodiment stimulates soothing sensation during or after activity to counter the pain. Optionally, the sleeve or other garment 12 may be made of a stretchable fabric so as to act as a compression garment. The combination of physical compression provided by the stretchable fabric and electrical stimulation of somatosensation is expected to provide a synergistic effect in alleviating pain from tennis elbow or other tendon issues, muscle cramping, or the like. In some embodiments, the fabric of the sleeve 12 providing a compression fit is an elastane fabric, such as spandex or lycra. Elastane fabrics comprise fibers of a long chain polyurethane, e.g. a polyether-polyurea copolymer.

In a similar and somewhat overlapping approach, the electrically applied somatosensations can produce pain gating using the stimulation. The pain gating operates on gate control theory, in which a non-painful stimulus closes nerve gates to pain signals. The electrically generated somatosensations can thus mask neuropathic pain from the arm, hand, lower back, neck, or other region experiencing pain. It is expected that high frequency stimulation will be particularly effective for blocking pain. The use of the sleeve or other garment 10 in which the array of electrodes 14 is embedded provides a wearable way to provide the pain gating stimulation. The garment is chosen based on the anatomy being treated: for example, a garment comprising shirt in which the array of electrodes 14 is embedded can provide stimulation to the lower back to alleviate lower back pain.

In a related application, the electrically applied somatosensations can block itch sensations, so as to quell the sensation of itch or simulate scratching to avoid injuring skin from too much scratching. This could be applied for therapy to humans, or in veterinary settings for pets, such as a dog or cat that is wearing a cone collar to prevent it from biting an injury or irritated area.

In another application, electrical stimulation of severed nerves after nerve damage has been shown to accelerate regrowth and guide reinnervation. For example, the array of electrodes 14 of the sleeve 12 can be used to apply spatially targeted somatosensation to guide nerve regeneration throughout the arm and hand. This allows the electrical stimulation to be applied to promote healing targeted specifically to anatomical regions that have become neurologically compromised.

In an analogous treatment, electrical stimulation provided by the array of electrodes 14 of the sleeve 12, optionally in combination with applied pressure and/or cooling, can help muscles to actively recover by promoting blood flow. Using the spatial resolution provided by the array of electrodes 14, the muscle groups can be targeted with electrical stimulation in a sequence of patterns to promote blood flow.

In another contemplated variant, applying an electrical stimulation pattern that evokes muscle contraction, thus performing functional electrical stimulation (FES), may be used to create a stronger haptic somatosensation for simulating an element in the VR or AR environment applying sufficient force to push the arm or other body part so as to cause the arm to move in response to the force.

In various nonlimiting embodiments disclosed as examples, a device comprises: an array of electrodes configured to be disposed on a body part; an electrical stimulation transmitter operatively coupled with the array of electrodes; and a data processing module including an electronic processor and a non-transitory storage medium storing spatiotemporal electrical stimulation patterns for generating corresponding somatosensations and further storing instructions readable and executable by the electronic processor to apply a spatiotemporal electrical stimulation pattern stored in the non-transitory storage medium using the electrical stimulation transmitter and the array of electrodes to generate the somatosensation corresponding to the applied spatiotemporal electrical stimulation pattern. In some specific various embodiments, the stored spatiotemporal electrical stimulation patterns may include patterns for generating a sensation of exposure of skin to a steam vent, a sensation of a weapon charge fire, a sensation of an athletic ring rotating around an arm, a sensation of raindrops falling on skin, a sensation of an arachnid crawling on skin, a sensation of a strike on a shield borne by an arm, and/or so forth.

The preferred embodiments have been illustrated and described. Obviously, modifications and alterations will occur to others upon reading and understanding the preceding detailed description. It is intended that the invention be construed as including all such modifications and alterations insofar as they come within the scope of the appended claims or the equivalents thereof.

1. A device comprising:
an array of electrodes configured to be disposed on a body part;
an electrical stimulation transmitter operatively coupled with the array of electrodes; and
a data processing module including an electronic processor and a non-transitory storage medium storing spatiotemporal electrical stimulation patterns for generating corresponding somatosensations and further storing instructions readable and executable by the electronic processor to apply a spatiotemporal electrical stimulation pattern stored in the non-transitory storage medium using the electrical stimulation transmitter and the array of electrodes to generate the somatosensation corresponding to the applied spatiotemporal electrical stimulation pattern.

2. The device of claim 1 wherein the stored spatiotemporal electrical stimulation patterns include a stored spatiotemporal electrical stimulation pattern for generating a sensation of exposure of skin to a steam vent.

3. The device of claim 1 wherein the stored spatiotemporal electrical stimulation patterns include a stored spatiotemporal electrical stimulation pattern for generating a sensation of at least one of a weapon charge fire and/or a strike on a shield borne by an arm.

4. The device of claim 1 wherein the stored spatiotemporal electrical stimulation patterns include a stored spatiotemporal electrical stimulation pattern for generating a sensation of an athletic ring rotating around an arm.

5. The device of claim 4 wherein the stored spatiotemporal electrical stimulation pattern for generating a sensation of an athletic ring rotating around an arm includes:
a first stored spatiotemporal electrical stimulation pattern for generating a sensation of an athletic ring rotating clockwise around an arm; and
a second stored spatiotemporal electrical stimulation pattern for generating a sensation of an athletic ring rotating counterclockwise around an arm.

6. The device of claim 1 wherein the stored spatiotemporal electrical stimulation patterns include a stored spatiotemporal electrical stimulation pattern for generating a sensation of raindrops falling on skin.

7. The device of any claim 6 wherein the stored spatiotemporal electrical stimulation pattern for generating a sensation of raindrops falling on skin includes a plurality of different stored spatiotemporal electrical stimulation patterns for generating sensations of raindrops falling on skin at different levels between light rainfall and heavy rainfall.

8. The device of claim 1 wherein the stored spatiotemporal electrical stimulation patterns include a stored spatiotemporal electrical stimulation pattern for generating a sensation of an arachnid crawling on skin.

9. The device of claim 1 wherein the stored spatiotemporal electrical stimulation patterns include a stored spatiotemporal electrical stimulation pattern for generating a spatially turning rectangular pulse simulating a sensation of a stab event.

10. The device of claim 1 wherein the array of electrodes includes at least 100 electrodes.

11. The device of claim 1 further comprising a garment in which the array of electrodes is embedded.

12. The device of claim 11 wherein the garment comprises a compression garment comprising a stretchable fabric.

13. The device of claim 1 wherein the stored spatiotemporal electrical stimulation patterns include a stored language of spatiotemporal electrical stimulation patterns for generating sensations corresponding to different notifications, and the device is connected to receive notifications from a mobile device and translate the received notifications into corresponding somatosensations of the stored language.

14. The device of claim 1 wherein the stored spatiotemporal electrical stimulation patterns include a stored high frequency stimulation providing pain gating.

15. A therapy method comprising:
disposing a garment on a body part experiencing pain, wherein an array of electrodes is embedded in the garment; and
with the garment disposed on the body part, applying an electrical stimulation pattern to the body part using the array of electrodes embedded in the garment.

16. The therapy method of claim 15 wherein the body part includes a ganglion cyst and the electrical stimulation pattern is applied to the gangion cyst.

17. The therapy method of claim 15 wherein the electrical stimulation pattern is applied to relieve tennis elbow, tendon-related pain, or muscle cramping.

18. The therapy method of claim 17 further comprising:
applying compression to the body part using the garment which comprises a compression garment made of a stretchable fabric.

19. The therapy method of claim 15 wherein the electrical stimulation pattern is applied to perform pain gating.

20. The therapy method of claim 15 wherein the pain is caused by nerve damage of the body part and the electrical stimulation pattern is applied in a pattern that accelerates and guides reinnervation.

21. The therapy method of claim 15 wherein the electrical stimulation pattern is applied to promote blood flow and the therapy method further comprises:
applying at least one of pressure and/or cooling to promote the blood flow.
